(19) United States
(12) Patent Application Publication
(10) Pub. No.: US 2026/0263133 A1
(43) Pub. Date: Sep. 10, 2026

(54) ENDOSCOPIC IRE APPARATUS AND METHOD FOR PANCREATIC CANCER PATIENT

(71) Applicant: UIF (UNIVERSITY INDUSTRY FOUNDATION), YONSEI UNIVERSITY, Seoul (KR)

(72) Inventors: Moon Jae CHUNG, Seoul (KR); Mandeuk KIM, Seoul (KR); Ho Kyoung HWANG, Seoul (KR); Seungmin BANG, Seoul (KR)

(73) Assignee: UIF (UNIVERSITY INDUSTRY FOUNDATION), YONSEI UNIVERSITY, Seoul (KR)

(21) Appl. No.: 19/658,117

(22) Filed: Apr. 24, 2026

Related U.S. Application Data
(63) Continuation of application No. PCT/KR2024/015247, filed on Oct. 8, 2024.

(30) Foreign Application Priority Data
Oct. 25, 2023 (KR) 10-2023-0143751

(51) Int. Cl. A61B 18/00; A61B 8/12

(52) U.S. Cl. CPC A61B 18/00; A61B 8/12; A61B 2018/00184; A61B 2018/00482; A61B 2018/00613; A61B 2018/00642; A61B 2018/00702; A61B 2018/0072; A61B 2018/00982

(57) ABSTRACT

The present disclosure may include an electrode module including at least one pair of needle-type electrodes inserted into a tumor mass of a pancreas; an energy supply module configured to selectively apply a bipolar-type current to the at least one pair of electrodes; a communication module configured to receive an image of the tumor mass acquired through an acquisition device; and a processor configured to control the energy supply module such that, when the at least one pair of electrodes reaches a target depth of the tumor mass based on the image of the tumor mass, a bipolar-type current is selectively applied to the at least one pair of electrodes with a current intensity corresponding to the reached target depth.

### CROSS-REFERENCE TO RELATED APPLICATIONS

The present application is a continuation of International Patent Application No. PCT/KR 2024/015247, filed on Oct. 8, 2024, which is based upon and claims the benefit of priority to Korean Patent Application No. 10-2023-0143751 filed on Oct. 25, 2023. The disclosures of the above-listed applications are hereby incorporated by reference herein in their entirety.

### BACKGROUND

### 1. Technical Field

The present disclosure relates to an endoscopic IRE apparatus. More specifically, the present disclosure relates to an endoscopic IRE apparatus and method for a pancreatic cancer patient.

### 2. Description of Related Art

Pancreatic cancer is a malignant tumor disease occurring in a pancreas, and is the 8th most common cancer, and a 5-year survival rate is only in a range of 10%.

Pancreatic cancer is a cancer for which a cause of occurrence is not specified, although a frequency of occurrence has recently increased by about 7 to 8% every year due to a change in dietary life and aging of a population.

Pancreatic cancer has almost no early symptoms, and thus early detection is difficult, and although a best treatment method is surgery, a possibility of surgery is low.

In pancreatic cancer, about 80 to 90% are patients with unresectable advanced or locally advanced pancreatic cancer at the time of diagnosis, and only radiation therapy and chemotherapy are available as existing standard treatments, and a recurrence possibility is higher than other types of cancer, and even a 5-year survival rate after surgery is very low at 20%, and since a prognosis is still poor with only the existing standard treatment method, an unmet need of patients and medical staff for a specific treatment option of pancreatic cancer is high.

In pancreatic cancer, it is impossible to improve a disease-related mortality rate through early detection and prevention as in cases of other malignant tumors at a current medical level, and about 90% of most patients are in a situation in which surgery is impossible at the time of diagnosis and a cure cannot be expected.

A case in which a condition is improved to a situation in which surgery is possible through such standard treatment is only within about 10%.

A conventional thermal treatment method has a high possibility of causing serious side effects by destroying not only cells but also surrounding sensitive tissues such as blood vessels and nerves by using thermal energy caused by radio frequency or laser.

In addition, cancer tissue around a vascular tissue due to heat sink of a blood vessel remains as it is, and thus a possibility of recurrence of cancer is high.

Accordingly, recently, research on a very safe and effective medical device for a patient with locally advanced pancreatic cancer in which surgical resection is impossible has been continuously conducted.

### SUMMARY

An embodiment disclosed in the present disclosure has an object to provide that can induce death of cancer cells while minimizing damage to sensitive tissues.

In addition, an embodiment disclosed in the present disclosure has an object to provide that can inhibit metastasis and growth of cancer tissue and secure time until chemotherapy is performed again.

In addition, an embodiment disclosed in the present disclosure has an object to provide that can reduce an administration amount of an anticancer agent by increasing a cell death rate.

Problems to be solved by the present disclosure are not limited to the problems mentioned above, and other problems not mentioned will be clearly understood by a person having ordinary skill in the art from the following description.

An endoscopic IRE apparatus for a pancreatic cancer patient according to one aspect of the present disclosure for achieving the above-described technical problem may include an electrode module including at least one pair of needle-type electrodes inserted into a tumor mass of a pancreas; an energy supply module configured to selectively apply a bipolar-type current to the at least one pair of electrodes; a communication module configured to receive an image of the tumor mass acquired through an acquisition device; and a processor configured to control the energy supply module such that, based on the image of the tumor mass, when the at least one pair of electrodes has reached a target depth of the tumor mass, a bipolar-type current is selectively applied to the at least one pair of electrodes with a current intensity corresponding to the reached target depth.

In addition, the electrode module may include four electrodes arranged at equal intervals.

In addition, a transfer module configured to move the electrode module such that the at least one pair of electrodes reaches a target depth of the tumor mass based on the image of the tumor mass may be further included.

In addition, the processor may determine, based on the image of the tumor mass, a distance between the at least one pair of electrodes, the target depth, a current intensity corresponding to the target depth, and a necrosis range and a necrosis shape of the tumor mass according to the current intensity.

In addition, the communication module receives a necrosis image of the tumor mass, and the processor may further control the energy supply module such that, based on the received necrosis image, a current intensity mapped to the necrosis image is determined and a bipolar-type current is selectively applied to the at least one pair of electrodes.

A method performed by an endoscopic IRE apparatus for a pancreatic cancer patient according to another aspect of the present disclosure may include receiving an image of a tumor mass; determining, based on the image of the tumor mass, whether at least one pair of electrodes has reached a target depth of the tumor mass; and controlling an energy supply module such that, when the at least one pair of electrodes has reached the target depth of the tumor mass, a bipolar-type current is selectively applied to the at least one pair of electrodes with a current intensity corresponding to the reached target depth.

In addition, an electrode module of the endoscopic IRE apparatus may include four electrodes arranged at equal intervals.

In addition, the method may further include moving an electrode module such that the at least one pair of electrodes reaches a target depth of the tumor mass based on the image of the tumor mass.

In addition, the controlling step may be determining, based on the image of the tumor mass, a distance between the at least one pair of electrodes, the target depth, a current intensity corresponding to the target depth, and a necrosis range and a necrosis shape of the tumor mass according to the current intensity.

In addition, the method may further include receiving a necrosis image of the tumor mass, and the controlling may be determining, based on the received necrosis image, a current intensity mapped to the necrosis image and controlling the energy supply module such that a bipolar-type current is selectively applied to the at least one pair of electrodes.

A pancreatic cancer treatment method using an endoscopic ultrasound according to another aspect of the present disclosure may include acquiring an image of a tumor mass of a pancreas by the endoscopic ultrasound; inserting a needle including a plurality of electrodes into the tumor mass based on the image acquired by the endoscopic ultrasound; inserting the needle such that at least one pair of electrodes among the plurality of electrodes included in the needle penetrates the tumor mass; and adjusting a current intensity of a bipolar-type current based on a size of the tumor mass and applying the bipolar-type current corresponding to the current intensity to the pair of electrodes.

In addition, the inserting of the needle may be moving the needle such that electrodes included in the needle reach a target depth of the tumor mass based on the image acquired by the endoscopic ultrasound.

In addition, the adjusting and applying step of the current intensity may be determining, based on the image acquired by the endoscopic ultrasound, a distance between the electrodes and a current intensity corresponding to a target depth of the tumor mass, and applying the bipolar-type current corresponding to the current intensity to the pair of electrodes.

In addition, the adjusting and applying of the current intensity may be receiving a necrosis image of the tumor mass acquired by the endoscopic ultrasound, and determining a current intensity based on the necrosis image and applying the bipolar-type current corresponding to the current intensity to the pair of electrodes.

In addition, a computer-readable recording medium recording a computer program for executing a method for implementing the present disclosure may be further provided.

### BRIEF DESCRIPTION OF THE FIGURES

FIG. 1 is a diagram illustrating an IRE system for a pancreatic cancer patient according to the present disclosure.

FIGS. 2 and 3 are diagrams illustrating a configuration of an endoscopic IRE apparatus for a pancreatic cancer patient of FIG. 1.

FIG. 4 is a flowchart illustrating a method of an endoscopic IRE apparatus for a pancreatic cancer patient according to the present disclosure.

FIGS. 5 and 6 are diagrams illustrating an operation process of an electrode module of FIG. 3.

FIGS. 7 to 11 are diagrams illustrating a process of removing a tumor mass of a pancreas using an endoscopic IRE apparatus based on an image of the tumor mass acquired through an acquisition device of FIG. 1.

FIG. 12 is a flowchart illustrating a pancreatic cancer treatment method according to the present disclosure.

FIG. 13 is a diagram illustrating a simulation result of an electric field distribution and a reference electric field distribution formed by a bipolar electrode according to the present disclosure.

FIG. 14 is a diagram illustrating a simulation result of a temperature distribution and a reference temperature distribution formed by a bipolar electrode according to the present disclosure.

FIGS. 15 and 16 are diagrams illustrating an electric field distribution state formed under an applied voltage condition with respect to a bipolar electrode arrangement of an endoscopic IRE apparatus according to the present disclosure.

### DETAILED DESCRIPTION

Throughout the present disclosure, the same reference numerals refer to the same components. The present disclosure does not describe all elements of the embodiments, and general content in the technical field to which the present disclosure belongs or content overlapping between embodiments is omitted. In the specification, terms such as ‘unit, module, member, block’ may be implemented as software or hardware, and according to embodiments, a plurality of ‘units, modules, members, blocks’ may be implemented as one component, or one ‘unit, module, member, block’may include a plurality of components.

Throughout the specification, when a part is “connected” with another part, it includes not only a case in which it is directly connected but also a case in which it is indirectly connected, and the indirect connection includes connection through a wireless communication network.

In addition, when a part “includes” a certain component, this means that other components are not excluded but may be further included unless particularly stated otherwise.

Throughout the specification, when a member is positioned “on” another member, this includes not only a case in which the member is in contact with the other member but also a case in which another member exists between the two members.

Terms such as first and second are used to distinguish one component from another component, and the components are not limited by the above terms.

A singular expression includes a plural expression unless there is a clear exception in the context.

Identification signs in each step are used for convenience of description, and the identification signs do not describe an order of the respective steps, and the respective steps may be performed differently from the stated order unless a specific order is clearly described in the context.

Hereinafter, a principle of operation and embodiments of the present disclosure will be described with reference to the accompanying drawings.

In the present specification, a controller of an endoscopic IRE apparatus for a pancreatic cancer patient according to the present disclosure includes all various devices capable of performing arithmetic processing and providing a result to a user. For example, the controller of the endoscopic IRE apparatus for a pancreatic cancer patient according to the present disclosure may include all of a computer, a server, and a portable terminal, or may be any one form.

Here, the computer may include, for example, a notebook equipped with a web browser, a desktop, a laptop, a tablet PC, a slate PC, and the like.

The server performs communication with an external device to process information, and may include an application server, a computing server, a database server, a file server, a mail server, a proxy server, and a web server.

The portable terminal is, for example, a wireless communication device in which portability and mobility are ensured, and may include all types of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, and a smart phone (Smart Phone), and may include wearable devices such as a watch, a ring, a bracelet, an anklet, a necklace, glasses, contact lenses, or a head-mounted-device (HMD).

An IRE system for a pancreatic cancer patient according to the present disclosure may receive an image of a tumor mass acquired through an acquisition device, determine whether at least one pair of electrodes has reached a target depth of the tumor mass based on the image of the tumor mass, and when the at least one pair of electrodes has reached the target depth of the tumor mass, control an energy supply module such that a bipolar-type current is selectively applied to the at least one pair of electrodes with a current intensity corresponding to the reached target depth.

Hereinafter, the IRE system for a pancreatic cancer patient will be described in detail.

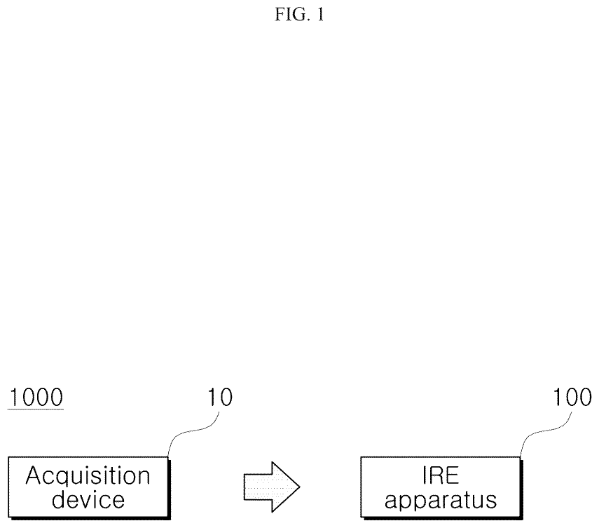

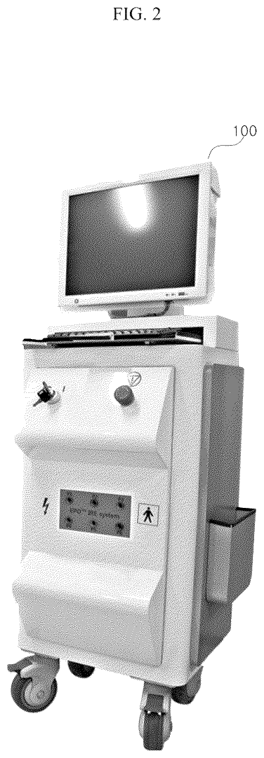

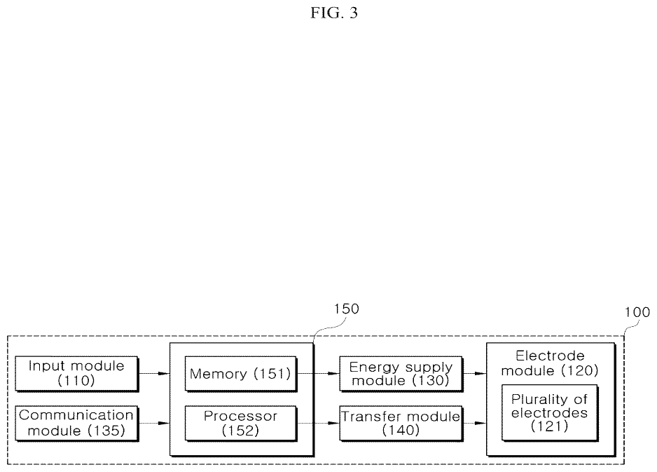

FIG. 1 is a diagram illustrating an IRE system for a pancreatic cancer patient according to the present disclosure. FIGS. 2 and 3 are diagrams illustrating a configuration of an endoscopic IRE apparatus for a pancreatic cancer patient of FIG. 1.

Referring to FIGS. 1 to 3, an IRE system 1000 for a pancreatic cancer patient may include an acquisition device 10 and an endoscopic IRE apparatus 100 for a pancreatic cancer patient.

The acquisition device 10 may acquire an image of a tumor mass of a pancreas. For example, the acquisition device 10 may be an endoscopic ultrasound (EUS), but is not limited thereto and may be any device capable of acquiring an image of a tumor mass of a pancreas.

The endoscopic IRE apparatus 100 for a pancreatic cancer patient may be an apparatus for a pancreatic cancer patient based on Irreversible electroporation (IRE). The Irreversible electroporation (IRE) is a technique that forms a plurality of pores in a cell membrane by using an electrode that delivers a high voltage of up to 3 kV into a tumor, and induces cell necrosis by damaging a homeostasis maintenance mechanism of a cell. In this case, the endoscopic IRE apparatus 100 for a pancreatic cancer patient may include an input module 110, an electrode module 120, an energy supply module 130, a transfer module 140, and a controller 150.

The input module 110 is for receiving target depth information of a tumor mass and energy irradiation condition information from a user, and when the target depth information of the tumor mass and the energy irradiation condition information are input, the controller 150 may control an operation of the apparatus to correspond to the input target depth information of the tumor mass and the energy irradiation condition information. Here, the target depth information of the tumor mass may be a target depth value of the tumor mass, a target depth value penetrating the tumor mass, or a target depth value adjacent to the tumor mass. In addition, the energy irradiation condition information may be conditions such as a size and a position of the tumor mass and a current intensity corresponding to the target depth.

The input module 110 may include a hardware-type physical key (e.g., a button, a dome switch, a jog wheel, a jog switch, etc. located on at least one of a front surface, a rear surface, and a side surface of the apparatus) and a software-type touch key. As an example, the touch key may be configured as a virtual key, a soft key, or a visual key displayed on a touch screen type display unit through software processing, or may be configured as a touch key disposed in a portion other than the touch screen. Meanwhile, the virtual key or the visual key may be displayed on the touch screen in various forms, and may be configured of, for example, a graphic, a text, an icon, a video, or a combination thereof.

The electrode module 120 may include at least one pair of needle-type electrodes inserted into a tumor mass of a pancreas. In this case, the electrode module 120 may include four electrodes arranged at equal intervals. The energy supply module 130 may selectively apply a bipolar-type current to the at least one pair of electrodes.

The communication module 135 may receive an image of the tumor mass acquired through the acquisition device 10. The communication module 135 may include at least one of a wired communication module and a wireless communication module. The wired communication module may include not only various wired communication modules such as a Local Area Network (LAN) module, a Wide Area Network (WAN) module, or a Value Added Network (VAN) module, but also various cable communication modules such as USB (Universal Serial Bus), HDMI (High Definition Multimedia Interface), DVI (Digital Visual Interface), RS-232 (recommended standard232), power line communication, or POTS (plain old telephone service). The wireless communication module may include, in addition to a Wifi module and a WiBro (Wireless broadband) module, a wireless communication module supporting various wireless communication schemes such as GSM (global System for Mobile Communication), CDMA (Code Division Multiple Access), WCDMA (Wideband Code Division Multiple Access), UMTS (universal mobile telecommunications system), TDMA (Time Division Multiple Access), LTE (Long Term Evolution), 4G, 5G, and 6G.

The transfer module 140 may move the electrode module 120 such that at least one pair of electrodes reaches a target depth of the tumor mass based on the image of the tumor mass. The transfer module 140 may move the electrode module 120 in a preset diagonal direction or a preset vertical direction.

The controller 150 may be implemented as a memory 151 storing data for an algorithm for controlling operations of components in the apparatus or a program reproducing the algorithm, and at least one processor 152 performing the above-described operations using the data stored in the memory 151. Here, the memory 151 and the processor 152 may each be implemented as separate chips. In addition, the memory 151 and the processor 152 may also be implemented as a single chip.

The memory 151 may store data supporting various functions of the apparatus, and a program for an operation of the controller, and may store input/output data, and may store a plurality of application programs (application program or application) driven in the apparatus, data for an operation of the apparatus, and instructions. At least some of these application programs may be downloaded from an external server through wireless communication.

The memory 151 may include a storage medium of at least one type among a flash memory type, a hard disk type, an SSD type (Solid State Disk type), an SDD type (Silicon Disk Drive type), a multimedia card micro type, a card-type memory (for example, SD or XD memory, etc.), RAM (random access memory), SRAM (static random access memory), ROM (read-only memory), EEPROM (electrically erasable programmable read-only memory), PROM (programmable read-only memory), a magnetic memory, a magnetic disk, and an optical disk. In addition, the memory 151 may be a database separated from the apparatus but connected in a wired or wireless manner.

The memory 151 may store data related to the endoscopic IRE apparatus 100 for a pancreatic cancer patient, and the processor 152 may control an operation related to the endoscopic IRE apparatus 100 for a pancreatic cancer patient.

The processor 152 may control the energy supply module 130 such that, based on the image of the tumor mass, when at least one pair of electrodes has reached a target depth of the tumor mass, a bipolar-type current is selectively applied to the at least one pair of electrodes with a current intensity corresponding to the reached target depth. In this case, the processor 152 may also determine, based on the image of the tumor mass, a distance between the at least one pair of electrodes, the target depth, a current intensity corresponding to the target depth, and a necrosis range and a necrosis shape of the tumor mass according to the current intensity.

The communication module 135 according to the present disclosure may receive a necrosis image of the tumor mass. In this case, the processor 152 may further control the energy supply module 130 such that, based on the received necrosis image, a current intensity mapped to the necrosis image is determined and a bipolar-type current is selectively applied to at least one pair of electrodes.

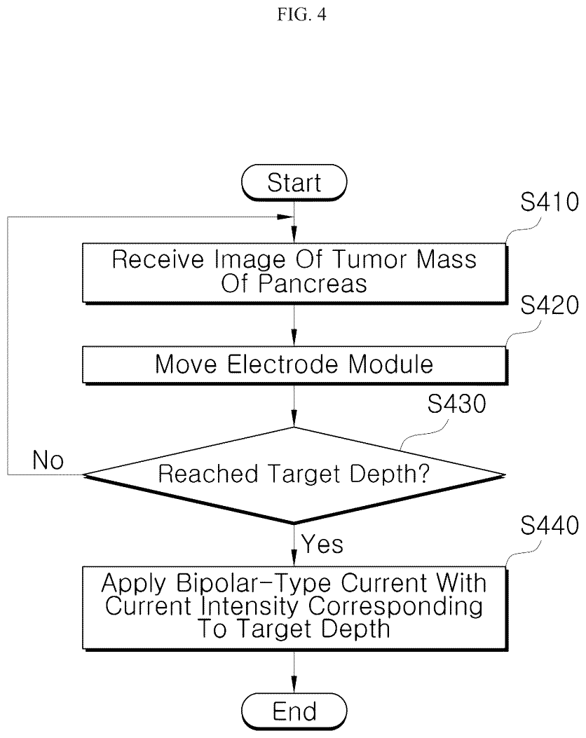

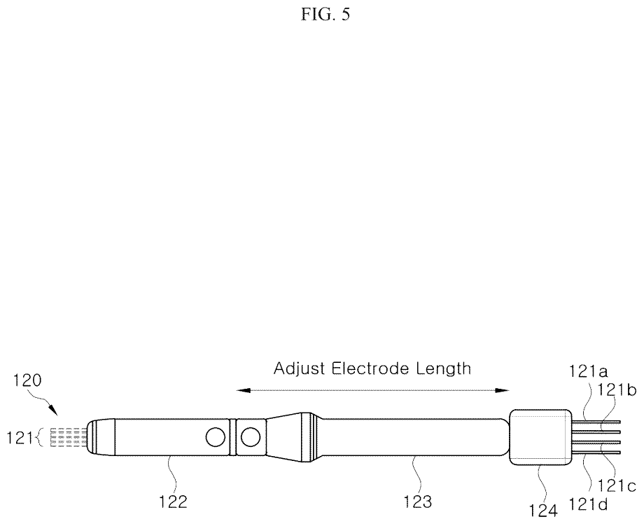

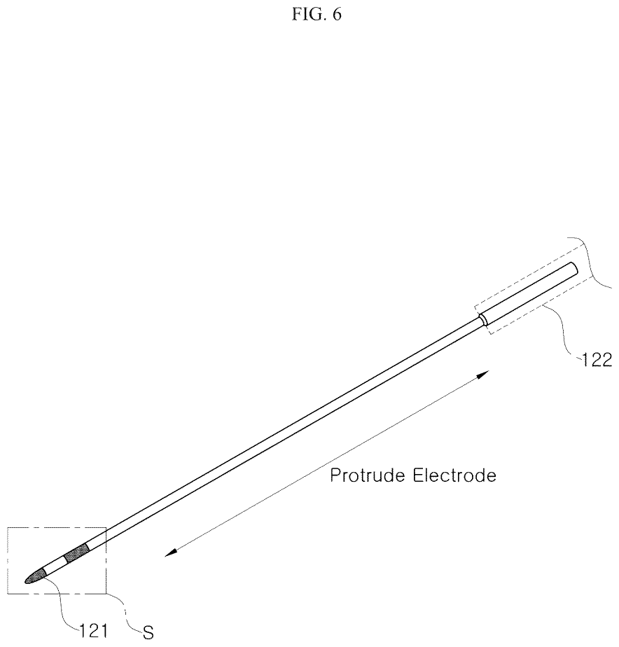

FIG. 4 is a flowchart illustrating a method of the endoscopic IRE apparatus for a pancreatic cancer patient according to the present disclosure. FIGS. 5 and 6 are diagrams illustrating an operation process of the electrode module of FIG. 3.

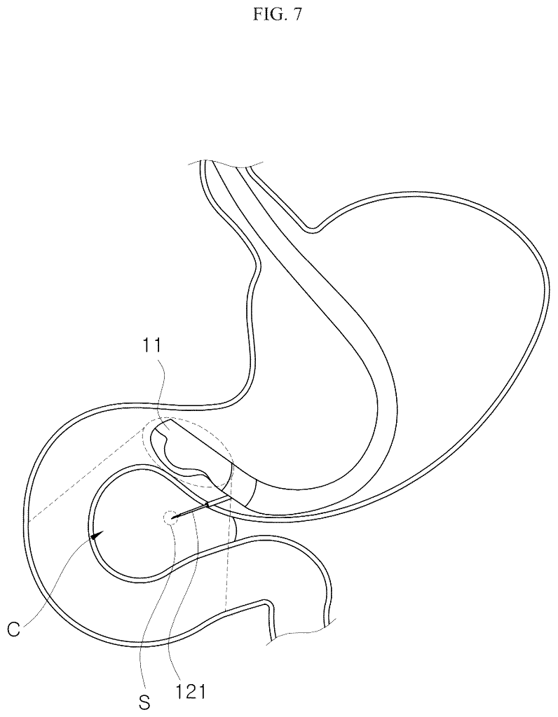

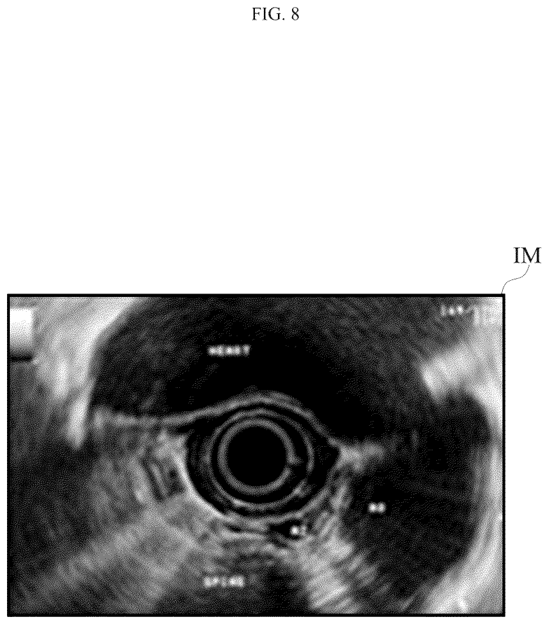

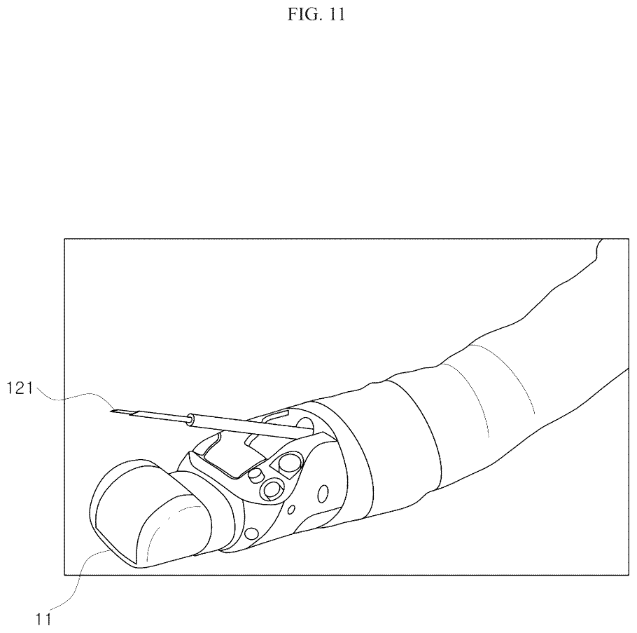

FIGS. 7 to 11 are diagrams illustrating a process of removing a tumor mass of the pancreas using the endoscopic IRE apparatus for a pancreatic cancer patient based on the image of the tumor mass acquired through the acquisition device of FIG. 1.

Referring to FIGS. 4 to 11, the method performed by the endoscopic IRE apparatus for a pancreatic cancer patient may include a receiving step S310, a moving step S320, a determining step S330, and a controlling step S340.

In the receiving step S310, the method performed by the IRE apparatus may receive an image of the tumor mass of the pancreas acquired from the acquisition device 10. For example, the acquisition device 10 may be an endoscopic ultrasound, but is not limited thereto and may be any device capable of acquiring an image of the tumor mass of the pancreas. In this case, the user may also input target depth information in the tumor mass and energy irradiation condition information by using the input module 110. Here, the target depth information in the tumor mass may be a target depth value in the tumor mass, a target depth value penetrating the tumor mass, or a target depth value adjacent to the tumor mass. In addition, the energy irradiation condition information may be conditions such as a size and a position of the tumor mass and a current intensity corresponding to the target depth.

In the moving step S320, the method performed by the IRE apparatus may move the electrode module 120 by controlling the transfer module 140 such that at least one pair of electrodes reaches a target depth of the tumor mass based on the image of the tumor mass. Here, as illustrated in FIGS. 5 and 6, the electrode module 120 may also include a first cover 122, a second cover 123, and an electrode connection module 124. In this case, the electrode connection module 124 may be detachably provided on the second cover 123, and four electrodes 121a to 121d arranged at equal intervals may be provided such that a length thereof is adjustable in an inner space of the first cover 122 and the second cover 123. For example, the four electrodes 121a to 121d and 121 arranged at equal intervals may protrude from the first cover 122.

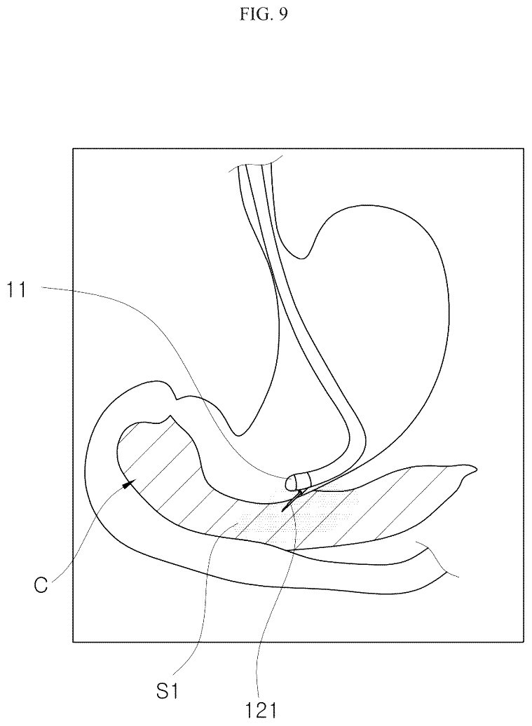

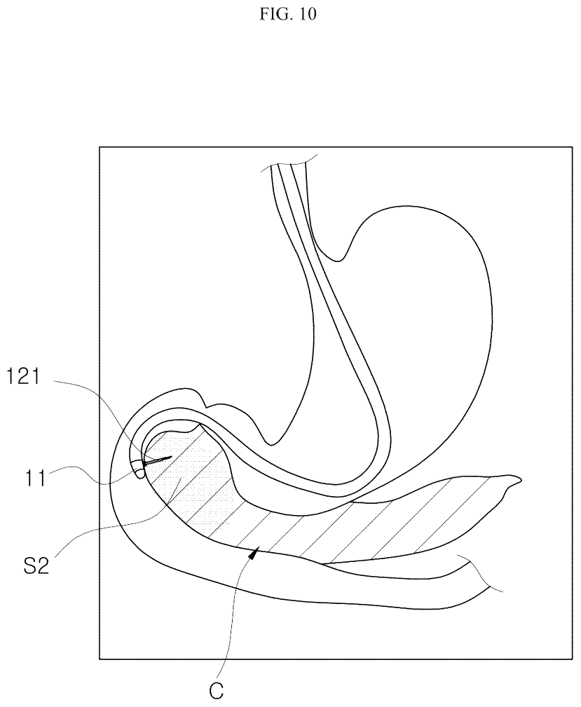

As illustrated in FIGS. 7 to 11, the user may check the image IM of the tumor mass S acquired through the endoscopic ultrasound 11, and may cause the protruding electrode 121 to penetrate into an interior of a body of the pancreatic cancer patient and reach a target depth of the tumor mass S in the pancreas C. In addition, as illustrated in FIGS. 9 and 10, the user may check the images of the tumor masses S1 and S2 acquired through the endoscopic ultrasound 11, and may cause the protruding electrode 121 to penetrate into the interior of the body of the pancreatic cancer patient and reach target depths of the tumor masses S1 and S2 in the pancreas C. In this case, at least one of the protruding electrodes 121a to 121d and 121 may penetrate the tumor mass S, S1, or S2.

In addition, the processor 152 may also control the transfer module 140 such that, based on the images of the tumor masses S, S1, and S2 acquired through the endoscopic ultrasound 11, the protruding electrode 121 penetrates into the interior of the body of the pancreatic cancer patient and reaches a target depth of the tumor masses S, S1, and S2 in the pancreas C. In this case, at least one of the protruding electrodes 121a to 121d and 121 may penetrate the tumor mass S, S1, or S2.

Here, the electrode module 120 may include at least one pair of needle-type electrodes inserted into the tumor masses S, S1, and S2 of the pancreas. According to one embodiment, the electrode module 120 may be configured of one pair of electrodes, that is, an electrode of (+) polarity and an electrode of (−) polarity, and one pair of electrodes may be inserted into the tumor masses S, S1, and S2 and a bipolar-type current may be applied.

According to another embodiment, the electrode module 120 may include two pairs of electrodes, that is, four electrodes 121a, 121b, 121c, and 121d arranged at equal intervals. Specifically, in the electrode module 120, the electrode 121a of (+) polarity and the electrode 121d of (−) polarity form a first pair, and the electrode 121b of (+) polarity and the electrode 121c of (−) polarity form a second pair, such that a bipolar-type current may be independently applied to each thereof.

In this case, the transfer module 140 may also move the electrode module 120 in a preset diagonal direction or a preset vertical direction.

In the determining step S330, the method performed by the IRE apparatus may determine, based on the image of the tumor masses S, S1, and S2, whether at least one pair of electrodes has reached a target depth of the tumor masses S, S1, and S2.

In the controlling step S340, the method performed by the IRE apparatus may control the energy supply module 130 such that, based on the image of the tumor masses S, S1, and S2, when at least one pair of electrodes has reached a target depth of the tumor mass, a bipolar-type current is selectively applied to at least one pair of electrodes with a current intensity corresponding to the reached target depth.

Here, the energy supply module 130 may selectively apply a bipolar-type current to a plurality of electrodes 121. In this case, the energy supply module 130 may include a switching circuit for selectively applying the bipolar-type current.

Meanwhile, in the present disclosure, according to a size and a position of a tumor, an applied voltage, a size of an electrode, a distance between electrodes, a current intensity, and an ablation shape and area may be changed. In addition, the present disclosure may also determine, based on the image of the tumor masses S, S1, and S2, a distance between at least one pair of electrodes, a target depth, a current intensity corresponding to the target depth, and a necrosis range and a necrosis shape of the tumor masses S, S1, and S2 according to the current intensity.

For example, the electrode module 120 may set, based on the image of the tumor masses S, S1, and S2, a distance between a plurality of electrodes 121a of (+) polarity and a plurality of electrodes 121d of (−) polarity, a distance between a plurality of electrodes 121b of (+) polarity and a plurality of electrodes 121c of (−) polarity, a distance between a plurality of electrodes 121b of (+) polarity and a plurality of electrodes 121d of (−) polarity, and a distance between a plurality of electrodes 121a of (+) polarity and a plurality of electrodes 121c of (−) polarity.

As another example, the electrode module 120 may set respectively, based on the image of the tumor masses S, S1, and S2, target depths according to a distance between a plurality of electrodes 121a of (+) polarity and a plurality of electrodes 121d of (−) polarity, a distance between a plurality of electrodes 121b of (+) polarity and a plurality of electrodes 121c of (−) polarity, a distance between a plurality of electrodes 121b of (+) polarity and a plurality of electrodes 121d of (−) polarity, and a distance between a plurality of electrodes 121a of (+) polarity and a plurality of electrodes 121c of (−) polarity. In this case, the processor 152 may determine, in order to accurately determine a necrotic site of the tumor masses S, S1, and S2, respective current intensities corresponding to the respective target depths, and a necrosis range and a necrosis shape of the tumor masses S, S1, and S2 according to the respective current intensities.

Therefore, the endoscopic IRE apparatus and method for a pancreatic cancer patient of the present disclosure can reduce side effects of an existing thermal minimally invasive procedure and a risk of recurrence of cancer, because compared to an existing thermal procedure, damage to a blood vessel, a nerve, and tissue and a heat sink effect are significantly low. In addition, a blood vessel or a bile duct structure within an IRE application range can be preserved because a surrounding supporting connective tissue matrix does not receive a heating effect by IRE application, and due to such a characteristic, it may become a very safe and effective apparatus for a patient with locally advanced pancreatic cancer in which surgical resection is impossible. That is, the endoscopic IRE apparatus for a pancreatic cancer patient of the present disclosure is efficient in removing a tumor adjacent to a sensitive tissue such as a blood vessel because heat generation is minimized as compared with a thermal treatment such as an existing radiofrequency ablation.

Meanwhile, in the controlling step S340, the method performed by the IRE apparatus may also receive a necrosis image of the tumor masses S, S1, and S2, and may control the energy supply module 130 such that, based on the received necrosis image of the tumor masses S, S1, and S2, a current intensity mapped to the necrosis image is determined and a bipolar-type current is selectively applied to at least one pair of electrodes. For example, the energy supply module 130 may selectively apply, while gradually lowering, a bipolar-type current to a plurality of electrodes 121a of (+) polarity and a plurality of electrodes 121d of (−) polarity, a bipolar-type current to a plurality of electrodes 121b of (+) polarity and a plurality of electrodes 121c of (−) polarity, a bipolar-type current to a plurality of electrodes 121b of (+) polarity and a plurality of electrodes 121d of (−) polarity, and a bipolar-type current to a plurality of electrodes 121a of (+) polarity and a plurality of electrodes 121c of (−) polarity, with a current intensity mapped to a preset necrosis image.

As another example, the energy supply module 130 may alternately apply, while gradually lowering, a bipolar-type current to a plurality of electrodes 121a of (+) polarity and a plurality of electrodes 121d of (−) polarity, a bipolar-type current to a plurality of electrodes 121b of (+) polarity and a plurality of electrodes 121c of (−) polarity, a bipolar-type current to a plurality of electrodes 121b of (+) polarity and a plurality of electrodes 121d of (−) polarity, and a bipolar-type current to a plurality of electrodes 121a of (+) polarity and a plurality of electrodes 121c of (−) polarity, with a current intensity mapped to a preset necrosis image.

As still another example, the energy supply module 130 may sequentially apply, while gradually lowering, a bipolar-type current to a plurality of electrodes 121a of (+) polarity and a plurality of electrodes 121d of (−) polarity, a bipolar-type current to a plurality of electrodes 121b of (+) polarity and a plurality of electrodes 121c of (−) polarity, a bipolar-type current to a plurality of electrodes 121b of (+) polarity and a plurality of electrodes 121d of (−) polarity, and a bipolar-type current to a plurality of electrodes 121a of (+) polarity and a plurality of electrodes 121c of (−) polarity, with a current intensity mapped to a preset necrosis image.

In this case, the processor 152 may control such that a preset rest interval is provided between application time points of a bipolar-type current to a plurality of electrodes 121a of (+) polarity and a plurality of electrodes 121d of (−) polarity, a bipolar-type current to a plurality of electrodes 121b of (+) polarity and a plurality of electrodes 121c of (−) polarity, a bipolar-type current to a plurality of electrodes 121b of (+) polarity and a plurality of electrodes 121d of (−) polarity, and a bipolar-type current to a plurality of electrodes 121a of (+) polarity and a plurality of electrodes 121c of (−) polarity. Here, the rest interval is an interval in which the bipolar-type current is not applied for a preset time, and the present disclosure can maximize an optimal energy irradiation rate of the bipolar type while preventing occurrence of a burn of the pancreas in advance by having the rest interval.

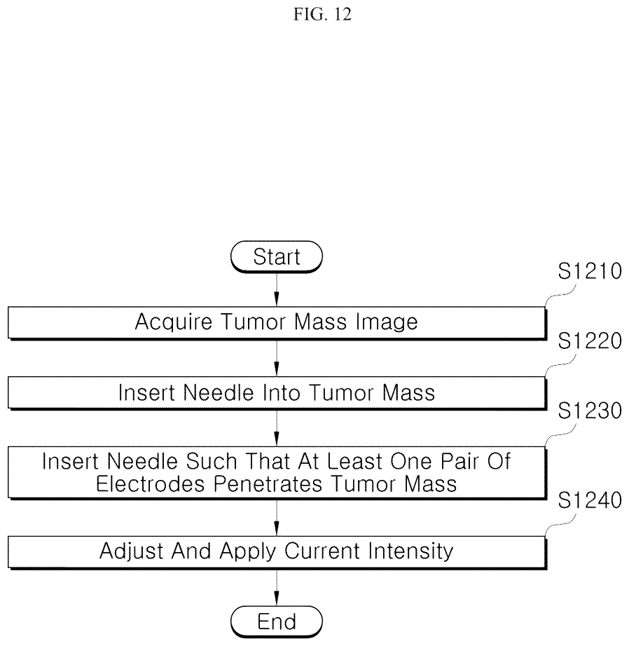

FIG. 12 is a flowchart illustrating a pancreatic cancer treatment method according to the present disclosure.

Referring to FIG. 12, the pancreatic cancer treatment method using the endoscopic ultrasound may include an acquiring step S1210, an inserting step S1220 and S1230, and an adjusting and applying step S1240.

In the acquiring step S1210, the pancreatic cancer treatment method may acquire an image of the tumor masses S, S1, and S2 of the pancreas by using the endoscopic ultrasound 11.

In the inserting step S1220, the pancreatic cancer treatment method may insert a needle including a plurality of electrodes 121 into the tumor masses S, S1, and S2 based on the image acquired by the endoscopic ultrasound 11. In this case, the needle may be inserted through an oral cavity or a transnasal route of the pancreatic cancer patient and may enter the tumor masses S, S1, and S2 located in the pancreas C. Such an insertion method of the needle may be performed in the same manner as an aspiration fine needle method, that is, EUS-FNA (Endoscopic Ultrasound-guided Fine Needle Aspiration) for collecting tissue under guidance of the endoscopic ultrasound. That is, the endoscopic IRE apparatus of the present disclosure may remove a tumor at a procedure level by approaching and inserting the needle into the tumor mass in the same manner as approaching a fine needle to the tumor in order to acquire tissue in an EUS-FNA procedure, without a separate surgical operation. Therefore, even for a patient in which surgical resection is impossible, the endoscopic IRE procedure can be safely performed by using the EUS-FNA procedure method already familiar to an operator as it is.

In the inserting step S1230, the pancreatic cancer treatment method may insert the needle such that at least one pair of electrodes among the plurality of electrodes 121 included in the needle penetrates the tumor masses S, S1, and S2. Specifically, the plurality of electrodes 121 may be moved to reach a target depth of the tumor masses S, S1, and S2, and accordingly, at least one electrode may penetrate the tumor masses S, S1, and S2 and may be positioned at both sides of the tumor.

In the adjusting and applying step S1240, the pancreatic cancer treatment method may adjust a current intensity of a bipolar-type current based on a size of the tumor masses S, S1, and S2, and may apply a bipolar-type current corresponding to the current intensity to one pair of electrodes.

As one example, in the adjusting and applying step S1240, the pancreatic cancer treatment method may determine, based on the image acquired by the endoscopic ultrasound 11, a distance between the electrodes and a current intensity corresponding to a target depth of the tumor masses S, S1, and S2, and may apply a bipolar-type current corresponding to the current intensity to one pair of electrodes.

As another example, in the adjusting and applying step S1240, the pancreatic cancer treatment method may receive a necrosis image of the tumor masses S, S1, and S2 acquired by the endoscopic ultrasound 11, and may determine a current intensity based on the necrosis image and apply a bipolar-type current corresponding to the current intensity to one pair of electrodes.

Accordingly, in a state in which the plurality of electrodes 121 are positioned at both sides of the tumor masses S, S1, and S2, a current corresponding to a size and a position of the tumor is applied, whereby irreversible electroporation for the tumor masses S, S1, and S2 may be induced.

Meanwhile, the pancreatic cancer treatment method may be performed in a manner in which a user checks an image of the tumor masses S, S1, and S2 of the pancreas by using the endoscopic ultrasound 11 while inserting a needle including a plurality of electrodes 121 through an oral cavity or a transnasal route of the pancreatic cancer patient and causing the needle to enter the tumor masses S, S1, and S2 located in the pancreas C. Such a procedure method may be performed in the same manner as an aspiration fine needle method, that is, EUS-FNA (Endoscopic Ultrasound-guided Fine Needle Aspiration) for collecting tissue under guidance of the endoscopic ultrasound. Specifically, in the same manner as obtaining tumor tissue with a fine needle during an EUS-FNA procedure, the endoscopic IRE apparatus of the present disclosure may approach the tumor mass through the endoscopic ultrasound and remove the tumor by inserting a needle electrode at a procedure level without a separate surgical operation. Accordingly, even for a patient with locally advanced pancreatic cancer in which surgical resection is impossible, an operator can perform the endoscopic IRE procedure more safely and conveniently by using an existing procedure method familiar in EUS-FNA as it is.

In this case, when the needle approaches the tumor masses S, S1, and S2, the user may insert the plurality of electrodes 121 such that the plurality of electrodes 121 penetrate the tumor masses S, S1, and S2 and may manipulate such that the plurality of electrodes 121 are positioned at both sides of the tumor masses S, S1, and S2, and at least one of the protruding electrodes 121a to 121d and 121 may penetrate the tumor masses S, S1, and S2.

When the plurality of electrodes 121 are positioned at both sides of the tumor masses S, S1, and S2, the processor 152 may determine a size of the tumor based on the image of the tumor masses S, S1, and S2 acquired through the endoscopic ultrasound 11, and may apply a bipolar-type current to the plurality of electrodes 121 by determining a current intensity corresponding to the size of the tumor and controlling the energy supply module 130, whereby irreversible electroporation for the tumor masses S, S1, and S2 may be induced.

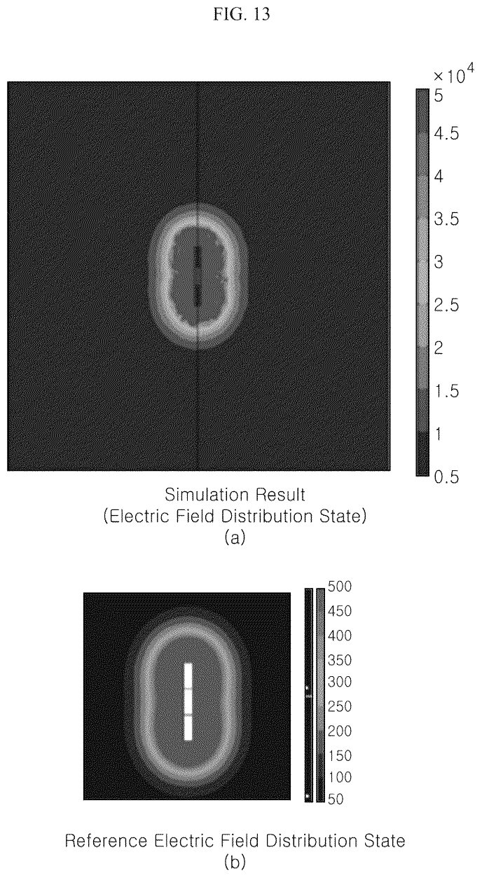

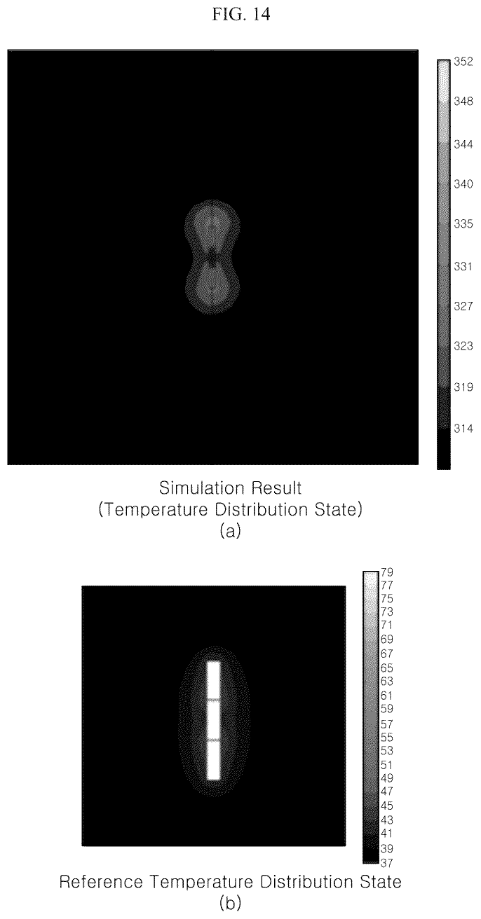

FIG. 13 is a diagram illustrating a simulation result of an electric field distribution and a reference electric field distribution formed by the bipolar electrode according to the present disclosure. FIG. 14 is a diagram illustrating a simulation result of a temperature distribution and a reference temperature distribution formed by the bipolar electrode according to the present disclosure.

Referring to FIGS. 13 and 14, numerical analysis results for an electric field distribution and a temperature distribution formed by a bipolar electrode arrangement provided in the endoscopic IRE apparatus according to the present disclosure were performed by using COMSOL Multiphysics, which is finite element method-based analysis software.

In this simulation, in setting electrical property values of pancreatic tissue, values reported in prior literature were applied as a reference range, and an electrical conductivity was set to 0.11 S/m and a temperature coefficient was reflected as 2.0%/°C. The electric field distribution was calculated by numerically analyzing the Laplace equation ∇·(σ∇φ)=0, and the temperature distribution was calculated by coupling the Pennes bioheat equation.

(a) of FIG. 13 is a simulation result illustrating an electric field distribution state formed by the bipolar electrode arrangement of the present disclosure. A right bar represents an electric field magnitude in a range of about 0.5×10⁴ V/m to 5×10⁴ V/m, and a color change means a spatial distribution of electric field intensity. According to the result, in an adjacent region of the electrodes, a high electric field of up to about 5×10⁴ V/m about 5,000 V/cm indicated by a red region is formed, and this is a result in which a potential gradient is intensively formed near a surface of the electrodes by an applied voltage between the electrodes. The electric field of the above level corresponds to a sufficient magnitude to generate irreversible electroporation by inducing a critical potential in a cell membrane.

In addition, over an entire region between the electrodes, an electric field corresponding to about 1×10⁴ V/m to 3×10⁴ V/m about 1,000 to 3,000 V/cm is widely distributed, and this is a level sufficiently exceeding a generally known reference range for inducing irreversible electroporation about 500 to 680 V/cm.

Accordingly, a condition for inducing uniform cell death is formed over the entire tissue positioned between the electrodes. Meanwhile, as going toward an outer side, a low electric field region of about 0.5×10⁴ V/m or less appears, and this means that the electric field rapidly decreases in an outer region of the electrodes.

(b) of FIG. 13 is a reference result illustrating a reference electric field distribution state for the electric field distribution. A right bar represents an electric field distribution in a range of about 50 V/cm to 500 V/cm, and in a central portion of the electrodes, an electric field of up to about 400 to 500 V/cm is formed, but as becoming spaced apart from the electrodes, a distribution gradually decreasing to a level of about 200 V/cm or less, and further 100 V/cm or less is shown. This is because a potential gradient becomes gentle as a current path is not concentrated in a specific region but diffuses over the entire tissue. As a result, a region corresponding to a reference range for inducing irreversible electroporation is limitedly formed in an adjacent portion of the electrodes.

In contrast, in the bipolar electrode arrangement of the present disclosure, even under the same voltage condition, an electric field is concentrated in an inner region between the electrodes by a closed-circuit path between the electrodes, and an electric field of about 1,000 V/cm or more is maintained over a wide region. In addition, a maximum electric field also rises to a level of about 5,000 V/cm, thereby forming remarkably high electric field intensity compared to the reference electric field distribution state.

Therefore, the bipolar electrode arrangement of the present disclosure has a characteristic in which, by concentrating the electric field at a higher level and more uniformly in the region between the electrodes than the reference electric field distribution state, a sufficient electric field is applied to tumor tissue while being formed such that the electric field rapidly decreases in a region outside the electrodes, so that diffusion of the electric field to surrounding tissue is structurally suppressed.

(a) of FIG. 14 is a simulation result illustrating a temperature distribution state formed by the bipolar electrode arrangement of the present disclosure. A right bar represents a temperature distribution in a range of about 314 K to 352 K, and a change of the right bar means a degree of temperature rise in tissue. According to the result, in an adjacent region of an exposed portion of the electrodes, a high temperature region of up to about 352 K (about 79° C.) is formed, and this is a result in which Joule heating is intensively generated near a surface of the electrodes where current density is concentrated. The above temperature range corresponds to a level at which protein denaturation and thermal damage of cells may occur, but the corresponding high temperature region is formed while being limited to a narrow region around the electrodes.

In addition, in a region slightly spaced apart from the electrodes, an intermediate temperature region corresponding to about 330 K to 340 K (about 57 to 67° C.) is formed, and this is a result in which heat generated in an adjacent portion of the electrodes is gradually decreased in a process of being transferred to a surrounding area by heat conduction.

In addition, in an outer region, a low temperature region of about 314 K to 320 K (about 41 to 47° C.) is formed, and this means that a temperature rise is limited by a heat removal by blood flow and a heat diffusion effect in tissue, as a state maintained at a level similar to a body temperature (about 310 K) or slightly increased.

(b) of FIG. 14 is a reference result illustrating a reference temperature distribution state. A right bar represents a temperature distribution in a range of about 37° C. to 79° C., and in a central portion of the electrodes, a high temperature region of about 75 to 79° C. is formed, while even in a region spaced apart from the electrodes, a temperature region of about 50 to 60° C. or more is distributed relatively widely. This is because Joule heating is generated over a wide region as a current path is not limited to a specific region but diffuses over the entire tissue. As a result, a temperature gradient is gently formed and a heat influence range is relatively expanded.

In contrast, the bipolar electrode arrangement of the present disclosure exhibits a characteristic in which, even under the same condition, a maximum temperature rise region is limited to an adjacent portion of the electrodes, and a high temperature region corresponding to about 352 K (79° C.) is locally formed, while at the same time, in a region spaced apart from the electrodes, a temperature rapidly decreases to about 320 K or less. Accordingly, a temperature gradient is sharply formed, and a range in which heat is diffused to an outside is structurally limited.

Due to such electric field and heat distribution characteristics, the endoscopic IRE apparatus according to the present disclosure is controlled such that, while inducing irreversible electroporation by forming an electric field of about 500 to 680 V/cm or more in tissue, a temperature rise of about 352 K level is limited to an adjacent portion of the electrodes.

Accordingly, while an electrical cell death effect is maintained, diffusion of a thermal damage region of 50° C. or more to surrounding tissue is suppressed, so that an electrical treatment environment applicable also to a lesion adjacent to a sensitive structure such as a blood vessel or a bile duct is implemented.

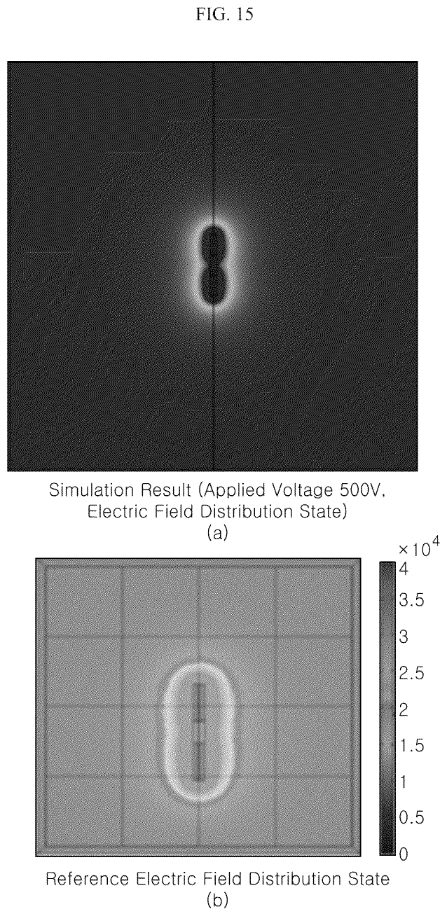

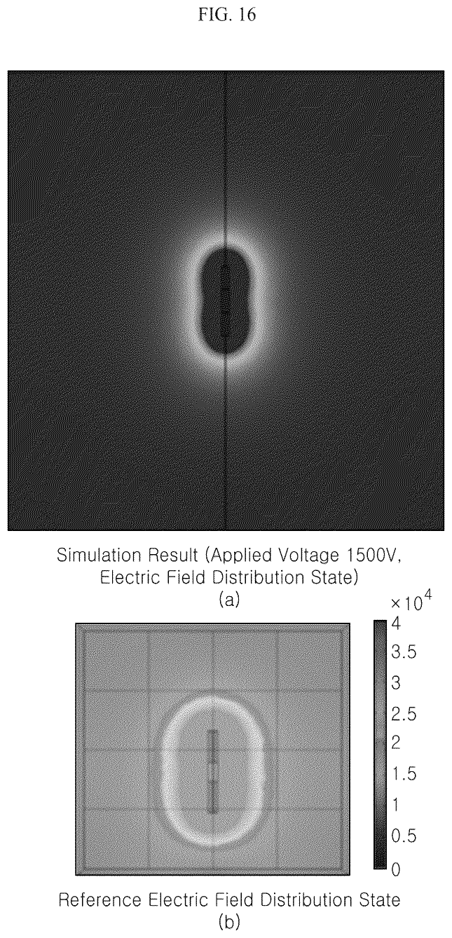

FIGS. 15 and 16 are diagrams illustrating an electric field distribution state formed under an applied voltage condition with respect to a bipolar electrode arrangement of the endoscopic IRE apparatus according to the present disclosure.

Referring to FIG. 15, the electric field distribution according to the present disclosure was performed by using COMSOL Multiphysics, which is finite element method-based analysis software, and was calculated in a state in which an electrode shape, a distance between electrodes, and electrical property values of tissue were reflected.

(a) of FIG. 15 is a simulation result illustrating an electric field distribution state formed by the bipolar electrode arrangement of the present disclosure under an applied voltage condition of 500 V. In an adjacent region of the electrodes, a high electric field corresponding to about 3×10⁴ V/m or more, up to about 4×10⁴ V/m about 3,000 to 4,000 V/cm was formed. This is a result in which a potential gradient between the electrodes is intensively formed in an adjacent region of electrode surfaces by an applied voltage of 500 V. In addition, over an entire region between the electrodes, an electric field of about 1×10⁴ V/m to 2×10⁴ V/m about 1,000 to 2,000 V/cm was distributed relatively uniformly, and this is a level sufficiently exceeding a reference range about 500 to 680 V/cm for inducing irreversible electroporation. On the other hand, in an outer region of the electrodes, an electric field of about 0.5×10⁴ V/m or less was formed, and this means that the electric field rapidly decreases toward an outside of the electrodes.

(b) of FIG. 15 is a reference result illustrating a reference electric field distribution state under the same 500 V condition. A right bar represents a range of about 0.5×10⁴ V/m to 4×10⁴ V/m, and in a central portion of the electrodes, an electric field of about 3×10⁴ V/m or more is formed, but as becoming spaced apart from the electrodes, the electric field gradually decreases and exhibits a tendency to diffuse to a level of about 1×10⁴ V/m or less. This is because a potential gradient becomes gentle as a current path is not limited between the electrodes but is distributed over the entire tissue.

In contrast, in the bipolar electrode arrangement of the present disclosure, even under the same 500 V condition, an electric field is concentrated in an inner region between the electrodes by a structure in which a closed-circuit path between the electrodes is formed, and an electric field of about 1,000 V/cm or more is maintained over a wide region. In addition, a maximum electric field is also formed at a level of about 3,000 to 4,000 V/cm, thereby exhibiting a characteristic in which higher electric field intensity is locally concentrated as compared with the reference electric field distribution state.

Therefore, the bipolar electrode arrangement of the present disclosure has a characteristic in which, even under the same voltage condition, an electric field is concentrated in a region between the electrodes at a higher level than in the reference electric field distribution state, and while applying a sufficient electric field to tumor tissue, the electric field is formed such that the electric field rapidly decreases in an outer region of the electrodes, whereby diffusion of the electric field to surrounding tissue is suppressed.

Referring to FIG. 16, the electric field distribution according to the present disclosure was performed by using COMSOL Multiphysics, which is finite element method-based analysis software, and was calculated in a state in which an electrode shape, a distance between electrodes, and electrical property values of tissue were reflected.

(a) of FIG. 16 is a simulation result illustrating an electric field distribution state formed by the bipolar electrode arrangement of the present disclosure under an applied voltage condition of 1500 V. In an adjacent region of the electrodes, a high electric field corresponding to about 3×10⁴ V/m or more, up to about 4×10⁴ V/m (about 3,000 to 4,000 V/cm) was formed. This is a result in which an electric field is concentrated near electrode surfaces while a potential gradient between the electrodes is formed more greatly by an applied voltage of 1500 V. In addition, over an entire region between the electrodes, an electric field corresponding to about 1×10⁴ V/m to 3×10⁴ V/m (about 1,000 to 3,000 V/cm) was distributed over a wide region, and this is a level sufficiently exceeding a reference range (about 500 to 680 V/cm) for inducing irreversible electroporation. Accordingly, an electrical environment in which cell membrane permeability is changed is formed in a wider region as compared with the 500 V condition. On the other hand, in an outer region of the electrodes, a low electric field region of about 0.5×10⁴ V/m or less was maintained, and this means that the electric field rapidly decreases toward an outside of the electrodes.

(b) of FIG. 16 is a reference result illustrating a reference electric field distribution state under the same 1500 V condition. A right bar represents a range of about 0.5×10⁴ V/m to 4×10⁴ V/m, and in a central portion of the electrodes, an electric field of about 3×10⁴ V/m or more is formed, but as becoming spaced apart from the electrodes, the electric field gradually decreases and exhibits a tendency to diffuse to a level of about 1×10⁴ V/m or less. This is because a current path is not limited between the electrodes but is distributed over the entire tissue. As a result, a high electric field region is spread relatively widely and an electric field gradient is gently formed.

In contrast, in the bipolar electrode arrangement of the present disclosure, even under the same 1500 V condition, an electric field is concentrated in an inner region between the electrodes by a closed-circuit path between the electrodes, and an electric field of about 1,000 V/cm or more was maintained more uniformly. In addition, while a maximum electric field was also formed at a level of about 3,000 to 4,000 V/cm, the corresponding high electric field region exhibited a characteristic limited to an adjacent portion of the electrodes.

Therefore, the bipolar electrode arrangement of the present disclosure has a characteristic in which, even under the same voltage condition, an electric field is formed in a more concentrated form than in the reference electric field distribution state, and while applying a sufficient electric field to tumor tissue, the electric field is maintained at a relatively low level in an outer region of the electrodes, whereby diffusion of the electric field to surrounding tissue is suppressed.

Meanwhile, when executed by one or more processors, a computer program stored in a computer-readable storage medium may perform the following operations for performing the method of the endoscopic IRE apparatus for a pancreatic cancer patient.

In this case, the computer program stored in the computer-readable storage medium may perform an operation of receiving an image of a tumor mass acquired through an acquisition device, and may perform, through the controller of the endoscopic IRE apparatus, an operation of determining, based on the image of the tumor mass, whether at least one pair of electrodes has reached a target depth of the tumor mass. In addition, the computer program stored in the computer-readable storage medium may also perform, through the controller, an operation of controlling the energy supply module of the endoscopic IRE apparatus such that, when at least one pair of electrodes has reached a target depth of the tumor mass, a bipolar-type current is selectively applied to at least one pair of electrodes with a current intensity corresponding to the reached target depth.

Here, according to another embodiment, the electrode module of the endoscopic IRE apparatus may also include two pairs of electrodes, that is, four electrodes arranged at equal intervals. In this case, the first pair of electrodes and the second pair of electrodes are independently controlled, so that electroporation can be performed on a wider tumor region.

In addition, the computer program stored in the computer-readable storage medium may also further perform an operation of moving the electrode module through the controller controlling the transfer module of the endoscopic IRE apparatus such that at least one pair of electrodes reaches a target depth of the tumor mass based on the image of the tumor mass. In this case, the control operation may determine, through the controller, based on the image of the tumor mass, a distance between at least one pair of electrodes, a target depth, a current intensity corresponding to the target depth, and a necrosis range and a necrosis shape of the tumor mass according to the current intensity.

In addition, the computer program stored in the computer-readable storage medium may further perform an operation of continuously receiving a real-time necrosis image of the tumor mass for a preset period through the communication module of the endoscopic IRE apparatus. In this case, the control operation may further control the energy supply module through the controller such that, based on the continuously received real-time necrosis image, a bipolar-type current is selectively applied to at least one pair of electrodes with a current intensity mapped to a preset real-time necrosis image.

Meanwhile, a computing device includes a processor including one or more cores, and includes a memory storing instructions executable by the processor, and when the instructions are executed by the processor, the processor may receive an image of a tumor mass acquired through an acquisition device, determine, based on the image of the tumor mass, whether at least one pair of electrodes has reached a target depth of the tumor mass, and when at least one pair of electrodes has reached a target depth of the tumor mass, control the energy supply module of the endoscopic IRE apparatus such that a bipolar-type current is selectively applied to at least one pair of electrodes with a current intensity corresponding to the reached target depth. In this case, according to another embodiment, the electrode module of the endoscopic IRE apparatus may also include two pairs of electrodes, that is, four electrodes arranged at equal intervals, and in this case, a first pair of electrodes and a second pair of electrodes may be independently controlled.

Here, the processor may further move the electrode module by controlling the transfer module of the endoscopic IRE apparatus such that at least one pair of electrodes reaches a target depth of the tumor mass based on the image of the tumor mass.

In addition, the processor may also determine, based on the image of the tumor mass, a distance between at least one pair of electrodes, a target depth, a current intensity corresponding to the target depth, and a necrosis range and a necrosis shape of the tumor mass according to the current intensity.

In addition, the processor may continuously receive a real-time necrosis image of the tumor mass for a preset period, and may further control the energy supply module such that, based on the continuously received real-time necrosis image, a bipolar-type current is selectively applied to at least one pair of electrodes with a current intensity mapped to a preset real-time necrosis image.

The present disclosure can minimize damage to sensitive tissue and can induce death of cancer cells through an endoscopic IRE apparatus using a bipolar electrode in which at least one pair of needle-type electrodes that can easily enter a pancreas is arranged, with respect to solid cancer such as locally advanced pancreatic cancer in which many sensitive tissues such as blood vessels are distributed around.

The present disclosure can inhibit metastasis and growth of cancer tissue and can secure time until chemotherapy is performed again by performing a method of the endoscopic IRE apparatus for a patient for whom it is difficult to continuously perform chemotherapy.

In the present disclosure, when a pore of a cell membrane is opened by using electroporation through the endoscopic IRE apparatus, an administration efficiency of an anticancer agent into a cell is increased, and thus a cell death rate is increased, so that an administration amount of the anticancer agent can be reduced. That is, in the present disclosure, through a high voltage of the endoscopic IRE apparatus, micro-perforation can be caused in a cell membrane of a tumor cell, and thus a death effect of the tumor cell can be expected, and furthermore, recovery of a patient is fast, so that additional administration of an anticancer agent is possible. In particular, the endoscopic IRE apparatus of the present disclosure is very useful in locally advanced pancreatic cancer because there is a possibility that an anti-tumor effect can be maximized through induction of immune activation of dead cells.

At least one component may be added or deleted in correspondence to performance of the components illustrated in FIGS. 1 to 3, FIGS. 5 to 11, and FIGS. 13 to 16. In addition, it will be easily understood by a person having ordinary skill in the art that mutual positions of the components may be changed in correspondence to performance or a structure of the system.

Although FIGS. 4 and 12 are described as sequentially executing a plurality of steps, this is only for illustratively describing the technical idea of the present embodiment, and a person having ordinary skill in the art to which the present embodiment belongs may variously modify and transform and apply the present embodiment by changing and executing the order described in FIGS. 4 and 12 or by executing one or more of the plurality of steps in parallel, within a range not departing from the essential characteristics of the present embodiment, and thus FIGS. 4 and 12 are not limited to a chronological order.

Meanwhile, the disclosed embodiments may be implemented in the form of a recording medium storing instructions executable by a computer. The instructions may be stored in the form of program code, and when executed by a processor, may generate a program module and perform operations of the disclosed embodiments. The recording medium may be implemented as a computer-readable recording medium.

The computer-readable recording medium includes all types of recording media in which instructions readable by a computer are stored. For example, there may be ROM (Read Only Memory), RAM (Random Access Memory), a magnetic tape, a magnetic disk, a flash memory, and an optical data storage device.

As described above, the disclosed embodiments have been described with reference to the accompanying drawings. A person having ordinary skill in the art to which the present disclosure belongs will understand that the present disclosure may be embodied in forms different from the disclosed embodiments without changing the technical idea or essential features of the present disclosure. The disclosed embodiments are illustrative and should not be construed in a limiting manner.

According to the above-described problem-solving means of the present disclosure, an effect of minimizing damage to sensitive tissue and inducing death of cancer cells is provided.

In addition, according to the above-described problem-solving means of the present disclosure, an effect of inhibiting metastasis and growth of cancer tissue and securing time until chemotherapy is performed again is provided.

In addition, according to the above-described problem-solving means of the present disclosure, since a cell death rate is increased, an effect of reducing an administration amount of an anticancer agent is provided.

Furthermore, according to the above-described problem-solving means of the present disclosure, by performing IRE treatment based on an image of a tumor mass acquired using an endoscopic ultrasound, electrodes can be inserted to a target depth of a tumor mass of a patient with locally advanced pancreatic cancer in which surgical resection is impossible and a current can be applied, and thus an effect of inducing electroporation for the tumor mass is provided.

What is claimed is:

1. An endoscopic IRE apparatus for a pancreatic cancer patient, comprising:
an electrode module comprising at least one pair of needle-type electrodes inserted into a tumor mass of a pancreas;
an energy supply module configured to selectively apply a bipolar-type current to the at least one pair of electrodes;
a communication module configured to receive an image of the tumor mass acquired through an acquisition device; and
a processor configured to control the energy supply module such that, based on the image of the tumor mass, when the at least one pair of electrodes has reached a target depth of the tumor mass, a bipolar-type current is selectively applied to the at least one pair of electrodes with a current intensity corresponding to the reached target depth.

2. The endoscopic IRE apparatus for a pancreatic cancer patient according to claim 1, wherein the electrode module comprises four electrodes arranged at equal intervals.

3. The endoscopic IRE apparatus for a pancreatic cancer patient according to claim 1, further comprising:
a transfer module configured to move the electrode module such that the at least one pair of electrodes reaches a target depth of the tumor mass based on the image of the tumor mass.

4. The endoscopic IRE apparatus for a pancreatic cancer patient according to claim 1, wherein the processor is configured to determine, based on the image of the tumor mass, a distance between the at least one pair of electrodes, the target depth, a current intensity corresponding to the target depth, and a necrosis range and a necrosis shape of the tumor mass according to the current intensity.

5. The endoscopic IRE apparatus for a pancreatic cancer patient according to claim 1, wherein,
the communication module is configured to receive a necrosis image of the tumor mass, and
the processor is configured to further control the energy supply module such that, based on the received necrosis image, a current intensity mapped to the necrosis image is determined and a bipolar-type current is selectively applied to the at least one pair of electrodes.

6. A method performed by an endoscopic IRE apparatus for a pancreatic cancer patient, comprising:
receiving an image of a tumor mass acquired from an acquisition device;
determining, based on the image of the tumor mass, whether at least one pair of electrodes has reached a target depth of the tumor mass; and
controlling an energy supply module such that, when the at least one pair of electrodes has reached the target depth of the tumor mass, a bipolar-type current is selectively applied to the at least one pair of electrodes with a current intensity corresponding to the reached target depth.

7. The method according to claim 6, wherein an electrode module of the endoscopic IRE apparatus comprises four electrodes arranged at equal intervals.

8. The method according to claim 6, further comprising:
moving an electrode module such that the at least one pair of electrodes reaches a target depth of the tumor mass based on the image of the tumor mass.

9. The method according to claim 6, wherein the controlling comprises:
determining, based on the image of the tumor mass, a distance between the at least one pair of electrodes, the target depth, a current intensity corresponding to the target depth, and a necrosis range and a necrosis shape of the tumor mass according to the current intensity.

10. The method according to claim 6, further comprising:
receiving a necrosis image of the tumor mass,
wherein the controlling comprises:
determining, based on the received necrosis image, a current intensity mapped to the necrosis image and controlling the energy supply module such that a bipolar-type current is selectively applied to the at least one pair of electrodes.

11. A pancreatic cancer treatment method using an endoscopic ultrasound, comprising:
acquiring an image of a tumor mass of a pancreas by the endoscopic ultrasound;
inserting a needle comprising a plurality of electrodes into the tumor mass based on the image acquired by the endoscopic ultrasound;
inserting the needle such that at least one pair of electrodes among the plurality of electrodes included in the needle penetrates the tumor mass; and
adjusting a current intensity of a bipolar-type current based on a size of the tumor mass and applying the bipolar-type current corresponding to the current intensity to the pair of electrodes.

12. The pancreatic cancer treatment method according to claim 11, wherein,
the inserting of the needle comprises:
moving the needle such that electrodes included in the needle reach a target depth of the tumor mass based on the image acquired by the endoscopic ultrasound.

13. The pancreatic cancer treatment method according to claim 11, wherein the adjusting and applying of the current intensity comprises:
determining, based on the image acquired by the endoscopic ultrasound, a distance between the electrodes and a current intensity corresponding to a target depth of the tumor mass, and
applying the bipolar-type current corresponding to the current intensity to the pair of electrodes.

14. The pancreatic cancer treatment method according to claim 11, wherein the adjusting and applying of the current intensity comprises:
receiving a necrosis image of the tumor mass acquired by the endoscopic ultrasound, and
determining a current intensity based on the necrosis image and applying the bipolar-type current corresponding to the current intensity to the pair of electrodes.
